(19) United States
(12) Patent Application Publication
(10) Pub. No.: US 2001/0043957 A1
(43) Pub. Date: Nov. 22, 2001

(54) Lypolytic composition

(76) Inventor: Morris Mann, Glendale, AZ (US)

Correspondence Address: The Halvorson Law Firm, Ste 1, 405 W. Southern Ave., Tempe, AZ, 85282, US

(21) Appl. No.: 09/781,775

(22) Filed: Feb. 12, 2001

Related U.S. Application Data
(60) Provisional application No. 60/184,818, filed on Feb. 25, 2000.

(51) Int. Cl. A61K 31/52; A61K 31/44; A61K 31/195; A61K 31/14; A61K 35/78

(52) U.S. Cl. 424/730; 424/725; 514/264; 514/290; 514/567; 514/642

(57) ABSTRACT

A composition for increasing lipolysis by enhancing oxygen transport is described. The composition comprises a thermogenic/lipolytic composition comprising a neurotransmitter replacement composition; an anti-inflammatory agent composition; an appetite suppressant composition; and a mineral replacement composition; wherein said thermogenic/lipolytic composition comprises at least one of the components selected from the group consisting of an Ephedra containing herb, a Theophylline extract, a Caffeine extract, and a Coleus Forskhollii extract; said neurotransmitter replacement composition comprises at least one of the components selected from the group consisting of an L-Tyrosine extract, Dimethylaminoethanol, Choline Citrate, and Huperzine-A; said anti-inflammatory agent composition comprises White Willow Bark; said appetite suppressant composition comprises at least one of the components selected from the group consisting of St. John's Wort and Passionflower Extract; and mineral replacement composition comprises at least one of the components selected from the group consisting of potassium, magnesium, and chromium salts.

### FIELD OF THE INVENTION

The present invention is generally directed to methods and compositions that enhance lipolysis and that suppress appetite in animal subjects, especially humans.

### BACKGROUND

Obesity continues to be a problem approaching pandemic proportions in this country. Unfortunately, most weight loss approaches utilize substances that are inherently tachyphylactic. Historically, substances that have induced weight loss have been amphetamines, their congeners, (phentermine, and the like) or their precursors (ephedra, ma huang, and the like). Unfortunately, these substances promote anxiety at therapeutic levels and with increased use their efficacy proportionately diminishes. These substances also promote a secondary depression upon withdrawal due to neurotransmitter depletion.

Thermogenic/lipolytic substances that promote lipolysis include a variety of different compounds and combinations that include, but are not limited to, ephedrine, xanthine compounds (such as caffeine and theophylline), and alpha adrenergic stimulants (Yohimbine, and the like) and beta adrenergic stimulants (clenbuterol, and the like). However, while these substances are thermogenic/lipolytic and produce lipolysis, they have no sustained effect on appetite suppression.

Interestingly, all of the aforementioned substances, including amphetamines, affect oxygen transport by increasing bronchial diameter. In effect, they are bronchodilators. They selectively affect relaxation of smooth muscle in the bronchial tree. The addition, therefore, of a substance that selectively relaxes smooth muscle would further enhance this effect. Surprisingly, alkaloids derived from coleus forskhollii synergistically enhance bronchodialation when combined with any of the aforementioned compounds (ephedrine, xanthines, and the like). This clearly enhances oxygen transport and therefore lipolysis.

Appetite suppression requires that neurotransmitters be in balance. This specifically includes the acetylcholine, serotonin, norepinephrine, and dopamine pathways. Imbalances in neurotransmitters can also cause both the symptoms of depression and anxiety. The vast majority of overeating occurs as a result of anxiety and/or depression. Regulating the neurotransmitter levels with appropriate supplementation will profoundly affect appetite and, therefore, sustained weight loss. Anxiolytic preparations, including passionflower and magnesium containing compounds, are particularly useful. Compounds that upregulate acetylcholine by reversibly inhibiting acetlylcholine breakdown are particularly effective. Physostigmine, pyridostigmine, and Huperzine-A are particularly effective. Adding choline containing compounds such as dimethylaminoethanol, phosphatidylcholine, and/or choline citrate will intensify the effect.

Finally, the addition of a substance that has antidepressant properties is a useful addition to any composition that suppresses appetite. St. John's Wort (hypericum) buproprion hydrochloride and S-adenosylmethionine are useful examples.

To supplement the body's ability to synthesize norepinephrine and dopamine, it is important to have the corresponding precursor amino acids tyrosine and/or phenylalanine. These substances will prevent the neurotransmitter depletion and secondary depression so often seen with the chronic administration of ephedrine and/or xanthine compounds.

Clearly, upon analysis, there exists a need in the art for a method of reducing appetite and increasing lipolysis. The method of the present invention would be the most opportune approach to engender sustained weight loss. The method and compositions related thereto will be evident upon reference to the following detailed description.

### SUMMARY OF THE INVENTION

The present invention provides an orally administered composition for enhancing lipolysis and inducing appetite suppression and methods related thereto. Surprisingly, it has been found that lipolysis and bronchodilation with the attendant increase in oxygen transport are intimately related.

It is another object of the present invention to produce a composition for creating weight loss benefits with herbal thermogenic/lipolytic products comprising containing ephedra, guarana, green tea and white willow bark, wherein said herbal thermogenic/lipolytic products must be properly combined in their formulation.

The novel features that are considered characteristic of the invention are set forth with particularity in the appended claims. The invention itself, however, both as to its structure and its operation together with the additional object and advantages thereof will best be understood from the following description of the preferred embodiment of the present invention. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. § 112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DESCRIPTION OF PREFERRED EMBODIMENTS

The present invention is generally directed to compositions and methods for enhancing lipolysis and suppressing the appetite. The purpose of this invention is to induce weight loss and then to sustain this effect. Although many specific details associated with certain aspects for the present invention are set forth below, those skilled in the art of pharmacology and especially neuro-pharmacology, will recognize that the present invention may have additional embodiments or that the invention may be practiced without several of the details disclosed herein.

The present invention provides an orally administered composition for enhancing lipolysis and inducing appetite suppression and methods related thereto. Surprisingly, it has been found that lipolysis and bronchodilation with the attendant increase in oxygen transport are intimately related.

If an individual wants to lose weight, reduce body fat, and improve muscle tone, more than diet is required. A total weight management and exercise program increases the body's metabolism and enables it to burn calories at a faster rate.

A diet simply limits the caloric intake. However, the body responds to the lower caloric intake by in turn lowering its metabolic rate in an attempt to conserve energy. Because at a lower metabolic rate the body is burning fewer calories than before, an individual gets disappointing weight loss results even though the caloric intake is lower. This is frequently true even for significant caloric reductions.

Exercise helps to increase the rate at which the body burns fat to thermogenic/lipolytically produce heat. This is why exercise, in addition to moderate daily caloric intake, has always been an effective means for weight loss. Now the medical community has discovered that herbal thermogens help boost the body's ability to burn fat and curb an individuals appetite, thus making the addition of herbal thermogens a great adjunct to exercise and diet programs.

Several products on the market contain the herb ephedra, a versatile thermogenic/lipolytic herb. Ephedra contains ephedrine, a stimulant naturally found in the plant. This herb promotes weight loss because it has a thermogenic/lipolytic and fat-metabolizing effect. Ephedrine also exhibits appetite suppressant properties by reducing one's desire for food. It is thought to activate both alpha and beta adrenoceptors, which elevates metabolic rate, thereby increasing caloric expenditure and resulting in weight loss. In other words, ephedrine simultaneously speeds up metabolism and reduces caloric intake by curbing the appetite.

It is important to remember that ephedra has been used for relieving asthma and allergies for many individuals. It is quite potent owing to the ephedrine and pseudoephedrine it contains. Both compounds excite the sympathetic nervous system, causing vasoconstriction of the nasal mucosa, dilation of the bronchioles, as well as cardiac stimulation. In the herb ephedra, these natural substances produce benefits similar to the body's adrenaline, but are not overly stimulating. This is why ephedra is such a useful herbal plant. It is extremely effective without being too strong in its actions when properly utilized.

The metabolic action of ephedra can be greatly enhanced when it is used in combination with a source of caffeine and xanthines. When ephedra is combined with guarana and green tea alone. Caffeine, methylxanthines, and ephedrine work together to to produce thermogenisis and to burn more fat than ephedrine alone. The greatest synergistic effect occurs when ephedra, guarana, green tea are combined with white willow bark (white willow bark is a source of salicin, a natural relative of aspirin). Some products designed to induce thermogenesis contain both ephedra and guarana plus occasionally white willow, but the fast acting synergistic combination of all four herbs yields the greatest results toward the goal of long lasting weight loss.

In order to produce the desired weight loss benefits, herbal thermogenic/lipolytic products containing ephedra, guarana, green tea and white willow bark must be properly combined in their formulation.

The potentiating action of caffeine and salicin (found in white willow bark) on ephedrine's action has been studied in numerous weight loss studies in animals and humans. A report in the American Journal of Clinical Nutrition showed that when ephedrine was used alone with a group of animals, it resulted in losses of 14 percent in body weight and 42 percent in body fat. When it was used in combination with caffeine, however, there was a loss of 25 percent in body weight and 75 percent in body fat. In contrast, when caffeine was used alone there was not significant loss in body weight. The reason for the increased loss of body weight is an increased metabolic rate and fat cell breakdown promoted by ephedrine and potentiated by caffeine.

In addition, one of the primary benefits of thermogenic/lipolytic formulas seems to be their ability to promote fat breakdown while sparing muscle tissue (since frequently low calorie diets also cause loss of muscle tissue). In one study reported by Astrup and coworkers in Metabolism (41:686-688, 1992), a combination of ephedrine (20 mg) and caffeine (200 mg), taken twice a day or a placebo for eight weeks in 16 obese women showed no significant difference in weight loss between groups. However, the ephedrine group lost on the average 4.5 kg more fat and 2.8 kg less muscle mass. While the total weight loss did not differ, the ephedrine group increased lipolysis while sparing muscle mass. Additionally, the ephedrine group had a higher level of energy expenditure than did the placebo group—a definite plus for dieting. The extra energy available for expenditure apparently resulted from the fat breakdown.

Although some authors have questioned the value of caffeine, Dr. Daniel Mowrey, in his book Fat Management! has proven caffeine to be effective in supporting the continued supply of neurotransmitters. Thus, caffeine acts not only to boost the stimulant effect for weight loss but to help preserve the balance of neurotransmitters necessary for total well being.

Acetylcholine is a neurotransmitter necessary for normal conduction of nerve impulses between nerve endings. In addition, the use of a reversible acetylcholinesterase inhibitor (a compound that inhibits the enzyme acetylcholinesterase, which breaks down acetylcholine and renders it ineffective at nerve junctions) further enhances acetylcholine levels and prevents attendant breakdown of this neurotransmitter level, in spite of chronic stimulant use. Huperzine A, a derivative of Chinese moss, is a particularly effective natural acetylcholinesterase inhibitor.

Caffeine is used in many cultures as a stimulant. Coffee is rapidly becoming our culture's “herbal” stimulant of choice. Studies show that most healthy people can safely consume up to 200 mg of caffeine and related methylxanthines per day without adverse reactions. Research shows that herbal thermogenic/lipolytic formulas are effective at levels well below this threshold.

Research reported by Dr. Dulloo and others in Nutrition (5:7-9, 1989), has shown that when caffeine is combined with lose-dose aspirin and ephedrine, an ephedra/kolaut/white willow bark mixture has been shown to cause significant weight loss in overweight persons who consume a reduced-calorie diet.

It is also known that when used in combination methylxanthines, caffeine, and ephedrine, all of which will induce bronchodilation, have the effect of increasing weight loss that is demonstrably better than when the compounds are used alone. Research has also shown that the herb coleus forskhollii also relaxes smooth muscle, thereby inducing or increasing bronchodilation. Surprisingly, the addition of this compound increases bronchodilation substantially in normal subjects, thereby increasing oxygen transport. This, in conjunction with the ability of caffeine to increase free fatty acid release will clearly result in an increased tendency towards lipolysis (the breakdown of fat).

It has been known for some time that small airway obstruction associated with clinical diseases such as asthma or emphysema have an inflammatory component. Surprisingly, the use of a known anti-inflammatory composition such salicylic acid (naturally found in white willow bark) in conjunction with bronchodilators also substantially increases oxygen transport, and consequently increases calorie burning.

It appears that the regular use of ephedra, caffeine, methylxanthines, coleus forskhollii and salicin in these amounts is relatively safe for most people. Ephedrine, especially in its herbal form, also appears relatively innocuous for individuals not in a high risk group. But caution needs to be exhibited by individuals with high blood pressure, heart disease, diabetes, those taking antidepressants, or certain other prescription or over-the-counter medications.

In addition to their thernoregulatory effects, ephedrine, caffeine, and theophylline (a xanthine found in green tea) have central nervous system stimulant effects. As a result two concerns arise with their continued use and when an individual stops taking these compounds. It is well known that taking these substances over a period of time can lead to a decrease in their response as a stimulant and a sub-threshold depression may occur with sustained use or abrupt withdrawal.

Specifically, the pathways for the neurotransmitters, acetylcholine, norepinephrine, and doparnine are affected. In order to ameliorate this problem, it is known that supplementation with neurotransmitter precursors can be effective in maintaining physiological levels of the neurotransmitters in the body.

The use of certain amino acids such as L-tyrosine has been found to be particularly important since these are precursors to norepinephrine and dopamine. The use of various choline containing compounds such as choline citrate, and dimethlyaminoethanol (DMAE), etc. have proven effective in supporting the continued supply of neurotransmitters.

In addition, the use of a reversible acetylcholinesterase inhibitor (a compound that inhibits the enzyme acetylcholinesterase, which breaks down acetylcholine and renders it ineffective at nerve junctions) further enhances acetylcholine levels and prevents attendant breakdown in spite of chronic stimulant use. Huperzine A, a derivative of Chinese moss, is a particularly good acetylcholinesterase inhibitor.

Huperzine A works by a unique mechanism that has been scientifically discovered and reported in many research journals. It acts as a potent acetylcholinesterase inhibitor. As stated earlier, acetylcholine is the neurotransmitter in the brain that is responsible for carrying electrical impulses from one nerve to another. Acetylcholine is produced in the end sections of nerve fibers and packaged into small vesicles where it is stored until released by the nerve ending. Once the nerve ending has secreted acetlycholine, it persists for a few seconds. In a normal brain, the enzyme acetylcholinesterase serves a housekeeping function by breaking down the acetylcholine into an acetate molecule (from the “acetyl” part) and choline. The choline (a member of the B-vitamin family) is then transmitted back into the nerve ending to be used again to make acetylcholine. Frequently when individuals take stimulant preparations, the precursors for producing acetylcholine are decreased, leading to a deficiency of acetylcholine available at the nerve junctions. Even with this deficiency, the acetylcholine is still released by the nerve endings. Huperzine A stops the acetylcholinesterase from breaking down acetylcholine, thus preventing acetylcholine deficiency and improving mental function.

Hypericum, the active ingredient in St. John's Wort, is known to be an effective anti-depressant and anti-anxiety agent (anxiolytic). Its effect is due to the inhibition of serotonin reuptake. Serotonin is another neurotransmitter whose levels affect mood, memory, anxiety and perceived energy levels. This, in conjunction with enhanced acetylocholine levels, surprisingly inhibits anxiety related to the use of any stimulant. Additionally, improvements in even sub-clinical depression and anxiety decrease the excessive appetite often seen with these conditions.

The amino acid L-tyrosine (normally present in dietary intake) is also important to controlling how the brain functions. The brain can use L-tyrosine to synthesize the neurotransmitters norepinephrine and dopamine, both of which are critical to the feeling of alertness and stability. The addition of L-tyrosine to the preparation assists the brain to stabilize levels of norepinephrine and dopamine that would otherwise be depleted by stimulant use.

The composition of the present invention involves several modules that are inherently interconnected, as will become obvious in the following description.

There are many known bronchodilators, some of which, such as ephedrine, are known to also be appetite suppressants. It was found that a combination of bronchodilators is better than any single compound alone. This has been known in clinical medicine for over 30 years. However, it was decided in this composition to use a combination of theophylline, caffeine, and ephedrine, all of which will induce bronchodilation. The effect of these compounds together is demonstrably better than any of the compounds alone. Although this is not particularly novel, described herein is the use of coleus forskholii, which as been found to relax smooth muscle. Surprisingly the addition of this compound increases bronchodilation substantially in normal subjects, thereby increasing oxygen transport. This, in conjunction with the ability of caffeine to increase free fatty acid release, will clearly result in an increased tendency towards lipolysis.

It has been known for some time that small airway obstruction associated with clinical diseases such as asthma or emphysema have an inflammatory component. Surprisingly, the use of a known anti-inflammatory composition, such as salicylic acid, acetylsalicylic acid, corticosteroids, and/or non-steriodal anti-inflammation drugs in conjunction with bronchodilators substantially increases oxygen transport as determined by measurement of the forced expiratory volume of air from the lungs in one second, termed FEV.

Ephedrine, caffeine, and theophylline all have central nervous system stimulant effects. As a result, it is well known that tachyphylaxis is a side effect of these stimulants and that sub-threshold depression may occur with sustained use or abrupt withdrawal. This is due to generalized neurotransmitter depletion. Specifically, the acetylcholine, norepinephrine, and dopamine pathways are affected. In order to ameliorate this problem, it was found that supplementation of neurotransmitter precursors was surprisingly effective. In addition, supplying an acetylcholinesterase inhibitor was particularly effective.

The use of certain amino acids such as L-phenylalanine and/or L-tyrosine was found to be particularly important since these are precursors to norepinephrine and dopamine. The use of various choline containing compounds including, but not limited to, phosphatidyl choline, choline citrate, dimethylaminoethanol, and the like, as proven effective. In addition, the use of a reversible acetylcholinesterase inhibitor further enhances acetylcholine levels and prevents attendant breakdown in spite of chronic stimulant use. Huperzine A, a derivative of Chinese moss, is a particularly good acetylcholinesterase inhibitor, although obviously other related pharmacological agents such as physostigmine or pyrodostigmine would also prove functional.

Anxiety and depression are known to result in excessive consumption of food beyond the body's nutritional requirements and dietary norms. Oftentimes, these conditions are sub-threshold; i.e., not clinically apparent. It is now apparent that overeating can be traced to deficiencies in certain neurotransmitters; i.e., norepinephrine and/or serotonin. Logically, therefore, substances that decrease and/or alleviate depression will be useful in preventing excessive dietary consumption.

Hypericum, the active ingredient in St. John's Wort, is known to be an effective anti-depressant. Its effect is due to the inhibition of serotonin reuptake. This, in conjunction with enhanced acetylcholine levels surprisingly inhibits appetite anxiety related to the use of any stimulation. Composition can be decreased with use of a variety of anxiolytic compounds. These include, but are not limited to, benzodiazepines, Cava alkaloids, passionflower, and/or chamomile extracts, and the like. Obviously, other antidepressants could be used such as buproprion hydrochloride, fluoxetine, and the like.

Several mineral compositions are useful in this formula. They include magnesium compounds such as magnesium phosphate or magnesium carbonate. It is known that magnesium and potassium contribute to the relaxation of smooth muscle. Additionally, chromium, in a variety of compositions, is also useful because of its known ability to regulate blood sugar and therefore reduce excess inappropriate appetite. Therefore, salts of these minerals are useful in the functioning of the formula.

A representative formula with ranges of ingredients in noted below:

| Ingredient | mg/capsule | | Range % |
|---|---|---|---|
| 1. Ephedra E @ 8% | 12 | mg | .1-40 |
| 2. Green Tea Extract @ 90% | 50 | mg | .1-40 |
| Theophylline | | | |
| 3. Guarana Extract @ 90% Caffeine | 40 | mg | .1-40 |
| 4. Coleus Forskholii extract @ 10% | 2.5 | mg | .001-20 |
| 5. L-Tyrosine | 65 | mg | .1-50 |
| 6. Dimethylaminoethanol | 50 | mg | .1-75 |
| 7. Choline Citrate | 50 | mg | .1-75 |
| 8. Huperzine-A | 0.009 | mg | .000001-5 |
| 9. St. John's Wort @ 0.3% hypericum | 25 | mg | .1-50 |
| 10. Passionflower Extract A | 30 | mg | .1-50 |
| 11. Potassium Chloride | 50 | mg | .1-20 |
| 12. Magnesium Phosphate Dibasic | 100 | mg | .1-80 |
| 13. Chromium Agrinate | 0.1 | mg | .001-20 |
| 14. White Willow Bark (Salicylic Acid) | 30 | mg | .01-75 |

| | |
|---|---|
| 15. Excipients | as necessary |

The first four ingredients are thermogenic/lipolytic compositions that effect smooth muscle relaxation in bronchioles. The second four ingredients are neurotransmitter replacements that prevent acetylcholine breakdown. St. John's Wort and Passionflower Extract A are appetite suppressants (as their central effect), the potassium, magnesium, and chromium salts are mineral replacements, and the White Willow Bark acts as an anti-inflammatory agent.

If indeed, bronchodilation and its attendant increase in oxygen transport are the cause for lipolysis, then relative increases in FEV will be directly related to weight loss. Surprisingly, this is indeed the case. Therefore, thermogenesis and resultant increases in metabolism are a direct consequence of increased oxygen delivery and consumption in either the resting or active state. To effectively demonstrate this principal, 10 individuals were given pulmonary function tests that measured FEV and weight was measured and recorded before administration of the composition as found in Example 1. The 10 individuals then regularly took the composition as found in Example 1. One month later, FEV and weight were re-measured. Interestingly, those with the largest positive increase in FEV (indicating the greatest amount of bronchodilation) consistently lost the most weight. See Table 1.

The preferred embodiment of the invention is described above in the Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1. A composition for creating weight loss benefits comprising
A. a thermogenic/lipolytic composition;
B. a neurotransmitter replacement composition; and
C. an anti-inflammatory agent composition.

2. The composition for creating weight loss benefits according to claim 1 further comprising an appetite suppressant composition.

3. The composition for creating weight loss benefits according to claim 1 further comprising a mineral replacement composition.

4. The composition for creating weight loss benefits according to claim 2 further comprising a mineral replacement composition.

5. The composition for creating weight loss benefits according to claim 1 wherein the thermogenic/lipolytic composition comprises at least one of the components selected from the group consisting of an Ephedra containing herb, a Theophylline extract, a Caffeine extract, and a Coleus Forskhollii extract.

6. The composition for creating weight loss benefits according to claim 1 wherein the neurotransmitter replacement composition comprises at least one of the components selected from the group consisting of an L-Tyrosine extract, Dimethylaminoethanol, Choline Citrate, and Huperzine-A.

7. The composition for creating weight loss benefits according to claim 6 wherein the neurotransmitter replacement composition comprises at least one of the components selected from the group consisting of an L-Tyrosine extract, Dimethylaminoethanol, Choline Citrate, and Huperzine-A.

8. The composition for creating weight loss benefits according to claim 1 wherein the anti-inflammatory agent composition comprises White Willow Bark.

9. The composition for creating weight loss benefits according to claim 5 wherein the anti-inflammatory agent composition comprises White Willow Bark.

10. The composition for creating weight loss benefits according to claim 6 wherein the anti-inflammatory agent composition comprises White Willow Bark.

11. The composition for creating weight loss benefits according to claim 2 wherein the appetite suppressant composition comprises at least one of the components selected from the group consisting of St. John's Wort and Passionflower Extract.

12. The composition for creating weight loss benefits according to claim 5 wherein the appetite suppressant composition comprises at least one of the components selected from the group consisting of St. John's Wort and Passionflower Extract.

13. The composition for creating weight loss benefits according to claim 6 wherein the appetite suppressant composition comprises at least one of the components selected from the group consisting of St. John's Wort and Passionflower Extract.

14. The composition for creating weight loss benefits according to claim 8 wherein the appetite suppressant composition comprises at least one of the components selected from the group consisting of St. John's Wort and Passionflower Extract.

15. The composition for creating weight loss benefits according to claim 4 wherein the mineral replacement composition comprises at least one of the components selected from the group consisting of potassium, magnesium, and chromium salts.

16. The composition for creating weight loss benefits according to claim 5 wherein the mineral replacement composition comprises at least one of the components selected from the group consisting of potassium, magnesium, and chromium salts.

17. The composition for creating weight loss benefits according to claim 6 wherein the mineral replacement composition comprises at least one of the components selected from the group consisting of po tassium, magnesium, and chromium salts.

18. The composition for creating weight loss benefits according to claim 8 wherein the mineral replacement composition comprises at least one of the components selected from the group consisting of potassium, magnesium, and chromium salts.

19. The composition for creating weight loss benefits according to claim 11 wherein the mineral replacement composition comprises at least one of the components selected from the group consisting of potassium, magnesium, and chromium salts.

20. A composition for creating weight loss benefits comprising
A. a thermogenic/lipolytic composition;
B. a neurotransmitter replacement composition;
C. an anti-inflammatory agent composition;
D. an appetite suppressant composition; and
E. a mineral replacement composition;
wherein said thermogenic/lipolytic composition comprises at least one of the components selected from the group consisting of an Ephedra containing herb, a Theophylline extract, a Caffeine extract, and a Coleus Forskhollii extract; said neurotransmitter replacement composition comprises at least one of the components selected from the group consisting of an L-Tyrosine extract, Dimethylaminoethanol, Choline Citrate, and Huperzine-A; said anti-inflammatory agent composition comprises White Willow Bark; said appetite suppressant composition comprises at least one of the components selected from the group consisting of St. John's Wort and Passionflower Extract; and mineral replacement composition comprises at least one of the components selected from the group consisting of potassium, magnesium, and chromium salts.
